(19) United States
(12) Patent Application Publication
(10) Pub. No.: US 2005/0288545 A1
(43) Pub. Date: Dec. 29, 2005

(54) Flexible tube for endoscope and method for manufacturing the same

(76) Inventors: Jun Matsumoto, Hino-shi (JP); Yasushi Machida, Hachioji-shi (JP); Takeaki Nakamura, Hino-shi (JP)

Correspondence Address: OSTROLENK FABER GERB & SOFFEN, 1180 AVENUE OF THE AMERICAS, NEW YORK, NY, 100368403, US

(21) Appl. No.: 11/095,360

(22) Filed: Mar. 31, 2005

(30) Foreign Application Priority Data
Mar. 31, 2004 (JP) 2004-107253
Aug. 25, 2004 (JP) 2004-245316
Sep. 14, 2004 (JP) 2004-267071

(51) Int. Cl. A61B 1/00

(52) U.S. Cl. 600/101

(57) ABSTRACT

A flexible tube for endoscope comprising a helical tube, a net tube put on the helical tube, an outer skin put on an outer peripheral surface of the net tube, and a topcoat put on a surface of the outer skin. The outer skin and the topcoat are molded substantially simultaneously or successively by the two-coat molding method.

### CROSS-REFERENCE TO RELATED APPLICATIONS

This application is based upon and claims the benefit of priority from prior Japanese Patent Applications No. 2004-107253, filed Mar. 31, 2004; No. 2004-245316, filed Aug. 25, 2004; and No. 2004-267071, filed Sep. 14, 2004, the entire contents of all of which are incorporated herein by reference.

### BACKGROUND OF THE INVENTION

1. Field of the Invention

The present invention relates to a flexible tube for endoscope, and more particularly, to a flexible tube for endoscope provided with a topcoat having good adhesion.

2. Description of the Related Art

Some conventional flexible tubes for endoscope is constituted by a helical tube, a net tube put the helical tube, a flexible outer skin covered on the net tube, and a topcoat coated on the outer skin. The topcoat lowers sliding friction resistance to facilitate insertion into a body cavity. The topcoat is formed by applying a resin solution to the flexible tube with the outer skin thereon and then drying and hardening it by heating. The flexible tube is coated by being dipped in the resin solution that is obtained by dissolving rubber-elastic urethane resin in a solvent. However, the coating by dipping causes variation in film thickness and requires heating and drying time after dipping, thereby entailing a long lead time.

There is known a flexible tube having an outer layer formed on an outer skin by extruding a thermoplastic fluoroelastomer (e.g., Jpn. Pat. Appln. KOKAI Publication No. 11-56762). However, the thermoplastic fluoroelastomer is expensive and its film is hard when hardened. If an insertion section of an endoscope is bent repeatedly, therefore, the outer layer may possibly peel off the outer skin.

Further, there is known a flexible tube having a silica coating layer obtained by converting perhydropolysilazane which is provided on the surface of an outer skin on a net tube (e.g., Jpn. Pat. Appln. KOKAI Publication No. 2000-93390). However, the silica coating layer is so hard that it easily peels off when it is disinfected with a chemical fluid, and its coefficient of friction is high. Thus, the insertion section of the endoscope cannot be easily inserted into a human body.

Another conventional flexible tube for endoscope is described in Jpn. Pat. Appln. KOKAI Publication No. 2000-107122. This flexible tube is formed by putting a net tube on a helical tube and then putting an outer skin on the outer peripheral surface of the net tube. This outer skin is formed by successively laminating a porous fluoroplastic layer, a fluororubber layer, and a fluorine containing coating layer to one another from inside to outside. The porous fluoroplastic layer and the fluororubber layer form an integrally molded tube, and fluororubber fills pores in the outer peripheral surface of the porous fluoroplastic layer like wedges.

The porous fluoroplastic layer is formed by sintering in a mold that is simultaneously biaxially centrifuged in horizontal and vertical directions. As this is done, voids are formed in unmelted portions between particles of fluoroplastic powder. These voids form continuous pores that penetrate the unmelted portions in all directions. Thus, the porous fluoroplastic layer is breathable.

In order to enable the insertion section of the endoscope to be inserted smoothly into the body cavity, the flexible tube may preferably have satisfactory resilience. However, the porous fluoroplastic layer is used in the flexible tube described in Jpn. Pat. Appln. KOKAI Publication No. 2000-107122, and fluoroplastic is not satisfactorily resilient. Further, the fluororubber layer is put on the porous fluoroplastic layer, and fluororubber fills the pores in the outer peripheral surface of the porous fluoroplastic layer like wedges. However, it is hard to obtain practically satisfactory resilience with this arrangement.

### BRIEF SUMMARY OF THE INVENTION

It is an object of the present invention to provide a flexible tube for endoscope, provided with a topcoat which is low-priced and reluctant to peel off, has a low coefficient of surface friction and a uniform thickness, and can be formed with ease.

It is another object of the invention to provide a flexible tube for endoscope which has high resilience.

According to a first aspect of the present invention, there is provided a flexible tube for endoscope, which comprises a helical tube, a net tube put on the helical tube, an outer skin put on an outer peripheral surface of the net tube, and a topcoat put on a surface of the outer skin, the outer skin and the topcoat being molded substantially simultaneously or successively by the two-coat molding method.

According to a second aspect of the present invention, there is provided a method for manufacturing a flexible tube for endoscope, which comprises spirally winding a thin elastic sheet to form a helical tube, putting a net tube on the helical tube, and welding and molding two types of resins substantially simultaneously on an outer peripheral surface of the net tube, thereby forming an outer skin and a topcoat welded together.

According to a third aspect of the present invention, there is provided a flexible tube for endoscope, which comprises a helical tube formed of a spirally wound belt member, a net tube put on the helical tube and formed of braided filament members, and an outer skin on the net tube, the outer skin having a foamed material layer.

The other aspects of the present invention are listed below.

1. A flexible tube for endoscope, which comprises an outer skin layer covering an outer peripheral surface of a tubular member and a topcoat covering an outer peripheral surface of the outer skin layer, the outer skin layer and the topcoat being molded by substantially simultaneously laminating resin materials together.

2. A flexible tube for endoscope, which comprises an outer skin layer covering an outer peripheral surface of a tubular member and a topcoat covering an outer peripheral surface of the outer skin layer, the outer skin layer and the topcoat being molded by successively laminating resin materials together.

3. A method for manufacturing a flexible tube for endoscope, which comprises laminating an outer skin layer of a resin material to an outer peripheral surface of a tubular member and laminating a topcoat of a resin material to an outer peripheral surface of the outer skin layer, laminating the outer skin layer and laminating the topcoat being performed substantially simultaneously.

4. A manufacturing method for a flexible tube for endoscope, which comprises laminating an outer skin layer of a resin material to an outer peripheral surface of a tubular member and laminating a topcoat of a resin material to an outer peripheral surface of the outer skin layer, laminating the outer skin layer and laminating the topcoat being performed successively.

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a side view showing a general configuration of an endoscope;

FIG. 2 is a sectional view showing a flexible tube for endoscope according to a first embodiment of the invention;

FIGS. 3 and 4 are side views showing manufacturing processes for the flexible tube for endoscope shown in FIG. 2;

FIG. 5 is a side view showing a molding machine used in the manufacturing processes shown in FIGS. 3 and 4;

FIG. 6 is a profile showing a process for preparing a helical tube and a net tube, among manufacturing processes for a flexible tube for endoscope according to a second embodiment of the invention;

FIG. 7 is a profile showing a process for applying an adhesive agent, among the manufacturing processes for the flexible tube for endoscope according to the second embodiment;

FIG. 8 is a profile showing a process for forming a foamed material layer, among the manufacturing processes for the flexible tube for endoscope according to the second embodiment;

FIG. 9 is a profile showing a process for forming a unfoamed material layer, among the manufacturing processes for the flexible tube for endoscope according to the second embodiment;

FIG. 10 is a profile showing the flexible tube for endoscope according to the second embodiment and a process for forming a coat layer, among the manufacturing processes for the flexible tube; and

FIG. 11 is a profile showing a flexible tube for endoscope according to a third embodiment of the invention and a process for forming a coat layer, among manufacturing processes for the flexible tube.

### DETAILED DESCRIPTION OF THE INVENTION

A flexible tube for endoscope according to a first embodiment of the present invention comprises a helical tube, a net tube put on the helical tube, an outer skin on the outer peripheral surface of the net tube, and a topcoat on the surface of the outer skin. The outer skin and the topcoat are molded substantially simultaneously or in succession by the two-coat molding method.

In the flexible tube for endoscope constructed in this manner, the topcoat that is molded by the two-coat molding method is provided on the outer surface of the outer skin. Therefore, the flexible tube has a smooth surface and high adhesive properties. Also, it can satisfactorily resist an autoclave sterilization process without peeling or cracking.

According to the two-coat molding method, both the outer skin and the topcoat are welded when they are molded simultaneously or the topcoat is molded with the molded outer skin at high temperature. Therefore, the outer skin and the topcoat can be securely welded together and their adhesion is extremely good. Thus, there may be obtained a flexible tube for endoscope that can satisfactorily resist the autoclave sterilization process without peeling or cracking of the topcoat.

A thermoplastic elastomer may be used as an available resin for the outer skin. The topcoat may be formed of a fully-saturated styrene-based thermoplastic elastomer, an elastomer obtained by copolymerizing a fully-saturated styrene-based thermoplastic elastomer and isobutylene, polyurethane, polyester, or the like. Since the thermoplastic elastomer has high heat resistance, wear resistance, and thermal weldability, it can be suitably used as the outer skin. On the other hand, the fully-saturated styrene-based thermoplastic elastomer is suited for the topcoat, since it is excellent in thermal degradation resistance, gas barrier properties, softness, and surface smoothness.

A method for manufacturing a flexible tube for endoscope according to a second embodiment of the invention comprises spirally winding a thin elastic sheet to form a helical tube, putting a net tube on the helical tube, and simultaneously welding and molding two resins on the outer peripheral surface of the net tube, thereby forming an outer skin and a topcoat that are welded together.

By providing the outer surface of the outer skin with the topcoat deposited on the outer skin, according to this method, there may be easily obtained a flexible tube for endoscope that enjoys a smooth surface and high adhesion and can satisfactorily resist the autoclave sterilization process without peeling or cracking.

The flexible tube for endoscope and its manufacturing method according to the first and second embodiments arranged in this manner have outstanding features as follows:

(1) Since the outer skin and the topcoat are welded together, the adhesion to the outer skin is so good that there is no possibility of the topcoat peeling off the outer skin or cracking if an insertion section of the endoscope is bent repeatedly.

(2) If the topcoat is formed of a fully-saturated styrene-based thermoplastic elastomer or an elastomer obtained by copolymerizing it and isobutylene, it enjoys high gas barrier. When the endoscope is subjected to the autoclave sterilization process, therefore, high-pressure vapor can never penetrate into the insertion section of the endoscope. Accordingly, there is no possibility of the outer skin being degraded or members in the insertion section of the endoscope being damaged.

(3) Since the outer skin and the topcoat can be molded simultaneously, the lead time can be reduced considerably, so that the flexible tube for endoscope can be manufactured at low cost.

A flexible tube for endoscope according to a third embodiment of the invention comprises a helical tube formed of a spirally wound belt member, a net tube put on the helical tube and formed of braided filament members, and an outer skin on the net tube, the outer skin having a foamed material layer.

The following is a description of a preferred or specific configuration of this flexible tube for endoscope.

1. An unfoamed material layer is formed on the outer peripheral surface of the foamed material layer.

2. The unfoamed material layer contains at least one selected from the group including polyester, polyurethane, polyolefin-based elastomer, styrene-based elastomer, fluorine-based elastomer, and silicone-based elastomer.

3. The foamed material contains a continuous cell, which is impregnated with an impregnant material having steam barrier properties.

4. The impregnant material contains at least one selected from the group including polyester, polyurethane, polyolefin-based elastomer, styrene-based elastomer, fluorine-based elastomer, and silicone-based elastomer.

5. The outer peripheral surface of the foamed material layer is fused by heating.

6. The outer skin includes isolated cell or a continuous cell.

7. The outer surface of the outer skin is molded by thermal fusion.

8. The continuous cell is impregnated with a resin.

9. The impregnant resin is highly resilient.

10. The impregnant resin is a steam-impermeable material.

The foamed material layer of the outer skin gives high resilience to the flexible tube for endoscope constructed in this manner.

The outer skin may include an unfoamed material layer that covers the outer peripheral surface of the foamed material layer. The unfoamed material layer on the foamed material gives steam barrier properties to the flexible tube for endoscope.

The unfoamed material layer may contain at least one selected from the group including polyester, polyurethane, polyolefin-based elastomer, styrene-based elastomer, fluorine-based elastomer, and silicone-based elastomer.

The foamed material layer may include a continuous cell, which may be impregnated with an impregnant material having steam barrier properties. This impregnation gives steam barrier properties to the foamed material layer.

The impregnant material may be at least one of materials including polyester, polyurethane, polyolefin-based elastomer, styrene-based elastomer, fluorine-based elastomer, and silicone-based elastomer.

The outer peripheral surface of the foamed material layer may be fused by heating. If the foam is collapsed by doing this, steam barrier properties are given to the flexible tube for endoscope.

Flexible tubes for endoscope according to various embodiments will now be described with reference to the accompanying drawings.

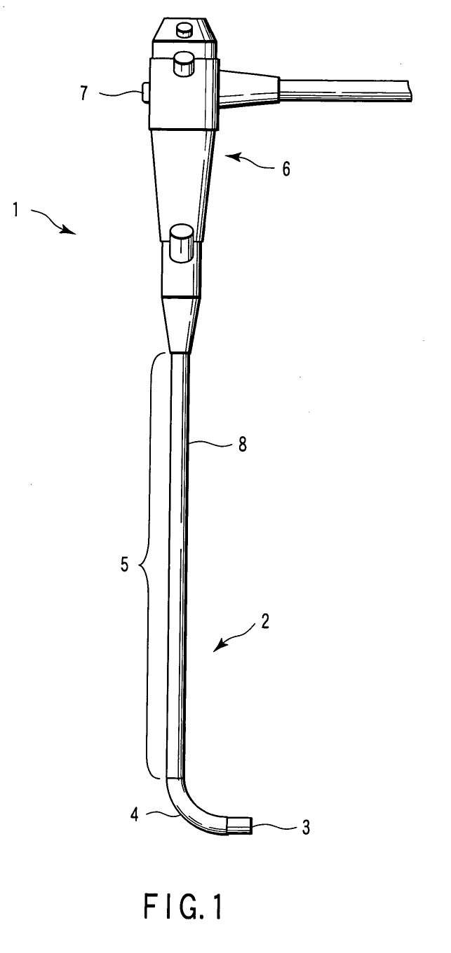

FIG. 1 shows a general configuration of an endoscope 1 that is provided with a flexible tube for endoscope according to an embodiment of the present invention. The endoscope 1 comprises an elongate insertion section 2 that can be inserted into a body cavity. The insertion section 2 is formed by successively coupling together a distal tip portion 3, a bending portion 4, and a flexible tube portion 5 from the distal end side. A control section 6 that can be held by an operator is provided on the proximal end portion of the insertion section 2. The control section 6 is provided with a control lever 7 for bending the bending portion 4.

The distal tip portion 3 is provided with a lens of an illumination optical system and an image-pickup device of an observation optical system, from which extend a light guide and a transmission cable. The light guide, transmission cable, and other contents are introduced into the control section 6 through the distal tip portion 3, bending portion 4, and flexible tube portion 5. The flexible tube portion 5 is formed of a flexible tube 8.

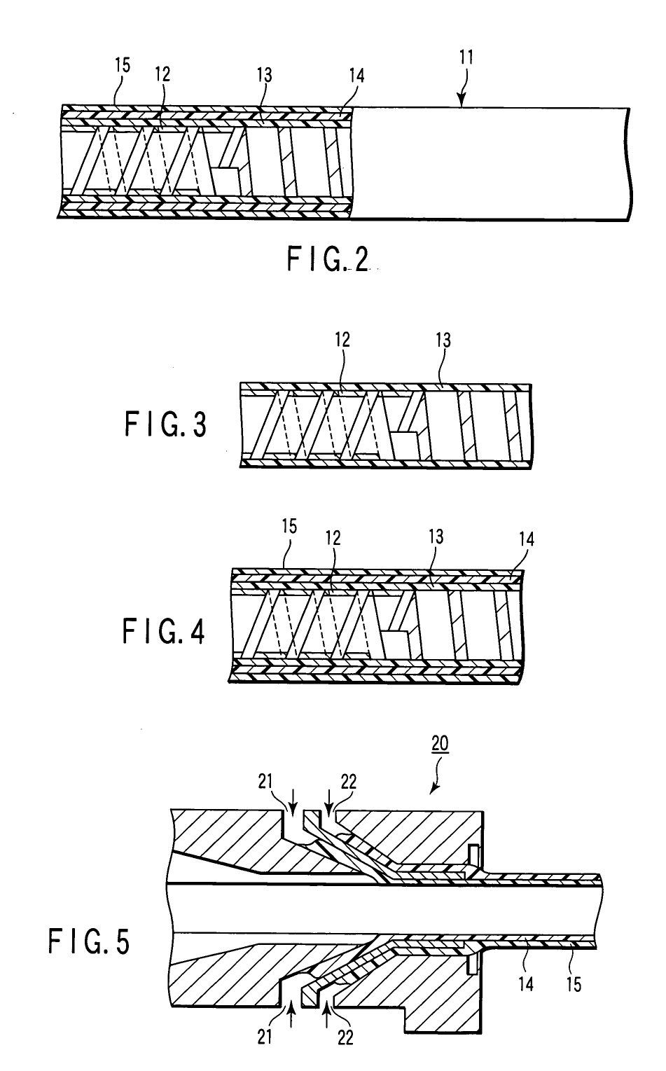

FIG. 2 is a sectional view showing a flexible tube 11 for endoscope according to a first embodiment of the invention.

As shown in FIG. 2, the flexible tube 11 is composed of a helical tube 12, a net tube 13 that covers the outer peripheral surface of the helical tube 12, an outer skin 14 that covers the outer peripheral surface of the net tube 13, and a topcoat 15 welded on the surface of the outer skin 14.

The helical tube 12 is formed by spirally winding a thin elastic sheet. Stainless steel or copper alloy may be used as a material for the thin elastic sheet. The net tube 13 is formed by braiding a plurality of metallic or nonmetallic filaments. Stainless steel or synthetic resin may be used as a metallic or nonmetallic material for the filaments. In some cases, the metallic and nonmetallic filaments may be mixedly braided together, in order to improve the adhesion to the outer skin resin.

Preferably, the material that forms the outer skin 14 on the outer peripheral surface of the net tube 13 may be a thermoplastic elastomer that has high heat resistance, wear resistance, and thermal weldability. The material that forms the topcoat 15 may preferably be a fully-saturated styrene-based thermoplastic elastomer that is excellent in thermal degradation resistance, gas barrier properties, softness, and surface smoothness. Combinations of these materials may be selected as required.

According to the present embodiment, the outer skin 14 and the topcoat 15 are molded by the two-coat molding method. The two-coat molding method is a method in which resins of two different types or colors are molded simultaneously or in succession by a molding machine that is provided with two molding devices. It is also named a two-material molding method. There are two molding methods, an extrusion or co-extrusion molding method and an injection or co-injection molding method.

According to the two-coat molding method, the two types of resins are molded simultaneously in a melted state, or one of the resins are molded with the other in a hot state immediately after its molding, so that the two resins can adhere to each other very firmly. Thus, this molding method is highly suited for molding the outer skin 14 and the topcoat 15 of the flexible tube.

Preferably, the thickness of the outer skin 14 ranges from 0.2 to 4 mm, and that of the topcoat 15 from 10 to 500 μm.

In the flexible tube 11 according to the present embodiment constructed in this manner, the outer skin 14 and the topcoat 15 are molding by the two-coat molding method, so that they are firmly welded together with good adhesion. If the topcoat 15 is repeatedly bent, cleaned, disinfected, or sterilized, therefore, it can never peel off the outer skin 14 or crack. Thus, when the endoscope is subjected to the autoclave sterilization process, there is no possibility of high-pressure steam from penetrating the insertion section of the endoscope through cracks. Since the topcoat 15 itself has gas barrier properties, moreover, the high-pressure steam can never get into the insertion section through the topcoat 15. In consequence, there is no possibility of the net tube 13 being degraded or the members in the insertion section being damaged.

The manufacturing method for the flexible tube for endoscope described above will now be described in the order of processes with reference to the accompanying drawings.

FIGS. 3 to 5 are sectional views showing manufacturing processes for the flexible tube for endoscope according to the first embodiment of the invention. First, as shown in FIG. 3, the net tube 13, which is obtained by braiding the filaments of, e.g., synthetic resin, like a net, is put on the helical tube 12, which is obtained by spirally winding a thin elastic sheet, such as a stainless steel sheet.

Then, an adhesive agent, such as a urethane-based adhesive (not shown), with which the net tube 13 can be easily impregnated, is applied to the outer peripheral surface of the net tube 13. Thereafter, two types of resins, e.g., polyester and the fully-saturated styrene-based thermoplastic elastomer, are extrusion-molded by the two-coat molding method. Thereupon, the outer skin 14 of 2-mm thickness and the topcoat 15 of 100-μm thickness are formed, as shown in FIG. 4.

The formation of the outer skin 14 and the topcoat 15 by the two-coat molding method is performed using a molding machine 20 shown in FIG. 5. More specifically, the molding machine 20 comprises a first nozzle 21 inside and a second nozzle 22 outside. Polyester is extruded from the first nozzle 21 onto the outer peripheral surface of the net tube 13, and the fully-saturated styrene-based thermoplastic elastomer, colorless or colored, is extruded from the second nozzle 22, whereupon the outer skin 14 and the topcoat 15 are molded simultaneously.

In order to finely adjust the flexibility of the flexible tube, the thermoplastic elastomer may be doped with any other resin that is compatible with the thermoplastic elastomer. A compatibilizer may be added to enhance the compatibility between the combined resins.

In consequence, the flexible tube 11 shown in FIG. 2 is obtained.

In the flexible tube for endoscope obtained in this manner, the melted topcoat 15 is molded on the melted outer skin 14, so that they can be firmly fixed to each other. If the flexible tube is repeatedly bent, it can never peel off the outer skin or crack. When the endoscope provided with this flexible tube was subjected to autoclave sterilization in a steam atmosphere of 135° C. and two atm. for five minutes, the high-pressure steam never penetrated the insertion section to degrade the net tube 13 or damage the members in the insertion section. Thus, the provided endoscope is safer in view of bacterial infection

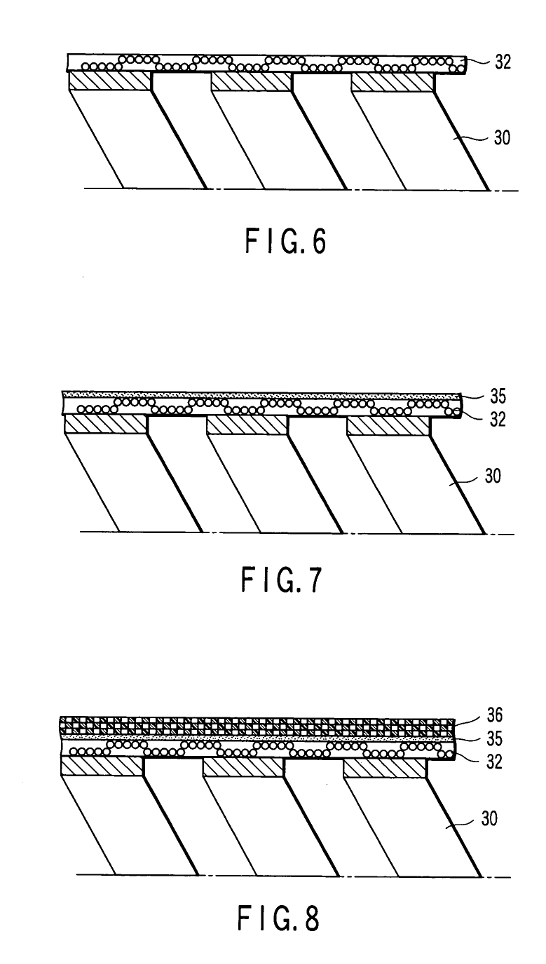

Manufacturing processes for a flexible tube 26 for endoscope according to the second embodiment of the invention will now be described with reference to FIGS. 6 to 10. The manufacturing processes include Steps 1 to 5, which are illustrated in FIGS. 6 to 10, respectively.

As shown in FIG. 6, a helical tube 30 is prepared in Step 1. The helical tube 30 is formed by spirally winding an elastic belt member to a fixed diameter. This belt member is formed of stainless steel or copper alloy, for example. A net tube 32 is fitted on the helical tube 30. It is formed of filaments or formed by braiding metallic and nonmetallic filaments. The metallic filaments are formed of stainless steel or copper alloy, for example. The nonmetallic filaments are formed of fibers of synthetic resin, such as polyester, polyamide, or polyvinyl chloride.

The following is a description of a manufacturing process for coating the net tube 32 with an outer skin 34 (see FIG. 10). In Step 2, a urethane-based adhesive agent 35, for example, is applied to the outer peripheral surface of the net tube 32, as shown in FIG. 7. In Step 3, a foamed material layer 36 is put on the outer peripheral surface of the adhesive agent 35, as shown in FIG. 8. The foamed material layer 36 is formed by coating the outer peripheral surface of the adhesive agent 35 with a thermoplastic elastomer that has good heat resistance, wear resistance, and adhesion to the adhesive agent 35 by extrusion molding. The foamed material layer 36 formed in this manner has high resilience.

Further, the foamed material layer 36 may include either isolated cells or a continuous cell. The foaming system may be chemical or mechanical foaming. In the chemical foaming, a foaming agent is previously mixed with a resin, and a gas is generated to form a foam by a chemical reaction of the foaming agent. In the mechanical foaming, a gas is externally compulsorily mixed into a resin to segmentalize a foam mechanically.

The Penguin Foam System (registered trademark), which is commercially available from Sunstar Engineering Inc., is a typical device for generating isolated cells by mechanical foaming. In the Penguin Foam System, a thermoplastic elastomer and air are fed into a foaming device to form a gas-liquid flow, which is then pumped. Air is finely dispersed into a thermoplastic elastomer, taking advantage of pumped flow characteristics. Thus, the thermoplastic elastomer is transferred under high pressure in a pipe as it is foamed.

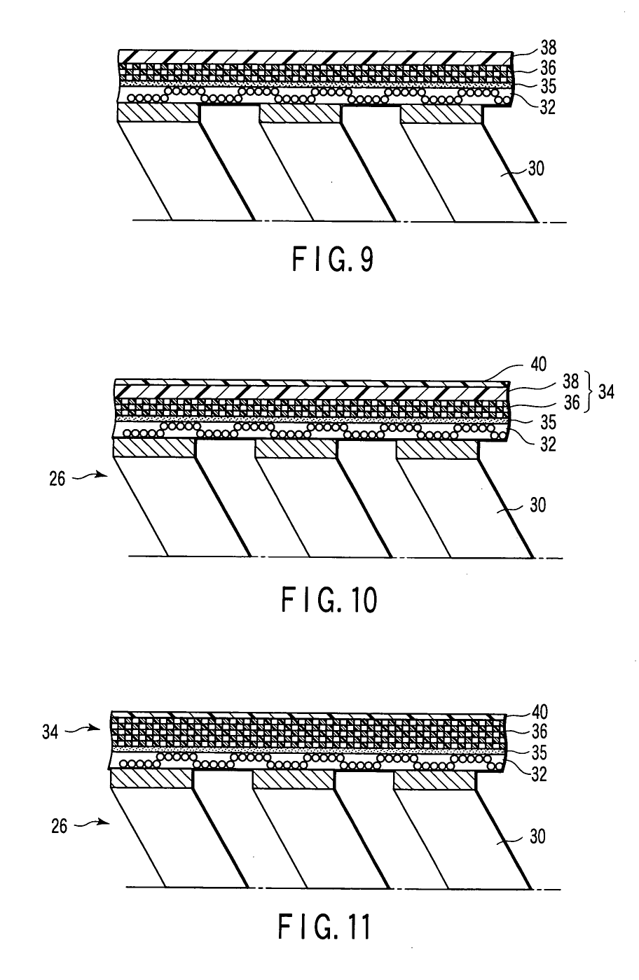

In Step 4, an unfoamed material layer 38 is formed on the outer peripheral surface of the foamed material layer 36, as shown in FIG. 9. The unfoamed material layer 38 is formed by attaching an unfoamed thermoplastic elastomer integrally to the outer peripheral surface of the foamed material layer 36. The unfoamed material layer 38 has steam barrier properties, and preferably, has high resilience. The unfoamed material layer 38 may be formed of, e.g., polyester, polyurethane, polyolefin-based elastomer, styrene-based elastomer, fluorine-based elastomer, or silicone-based elastomer.

In Step 5, a coating layer 40 is formed on the outer peripheral surface of the unfoamed material layer 38, as shown in FIG. 10. The coating layer 40 is formed by coating the outer peripheral surface of the unfoamed material layer 38 with a material that has high chemical resistance and slip characteristics, e.g., urethane- or fluorine-based resin, by dipping or extrusion molding. Thus, the flexible tube 26 shown in FIG. 10 is formed.

The following is a description of functions and effects of the endoscope that is provided with the flexible tube 26 constructed in this manner. In inserting the insertion section 2 of the endoscope 1 of FIG. 1 into the body cavity, the flexible tube 26 fulfills its high resilience that is based on the foamed material layer 36, a thermoplastic elastomer foam.

Further, the endoscope must be securely disinfected or sterilized after use. In some cases, autoclave sterilization that utilizes high-temperature, high-pressure steam may be used for the disinfection or sterilization, in consideration of the ease of operation, low running cost, etc. In subjecting the endoscope 1 to the autoclave sterilization, the unfoamed material layer 38, a thermoplastic elastomer, fulfills its steam barrier properties. Accordingly, the high-temperature, high-pressure steam hardly gets into the endoscope 1, so that the contents of the endoscope can be prevented from being degraded. Thus, the endoscope 1 can deal with the autoclave sterilization.

Furthermore, the adhesive agent 35 is applied to the outer peripheral surface of the net tube 32, and the foamed material layer 36 is formed by coating the outer peripheral surface of the adhesive agent 35 with a thermoplastic elastomer foam by extrusion molding. Thus, the foamed material layer 36 can be securely bonded to the net tube 32.

The following is a description of a flexible tube for endoscope according to the third embodiment of the invention. The same reference numerals are used to designate similar portions of the second and third embodiments, and a description of those portions is omitted. Steps 1 to 3 of manufacturing processes for a flexible tube 26 according to the present embodiment are equivalent to Step 1 to 3, respectively, of the second embodiment. More specifically, a net tube 32 is fitted on a helical tube 30, a adhesive agent 35 is applied to the net tube 32, and a foamed material layer 36 is put on the adhesive agent 35. The foamed material layer 36 is a continuous cell.

In Step 4, the continuous cell of the foamed material layer 36 is impregnated with an impregnant material that has steam barrier properties. In consequence, steam barrier properties are given to the foamed material layer 36. Preferably, the impregnant material has high resilience so that the resilience of the foamed material layer 36 is enhanced. The impregnant material may be, e.g., polyester, polyurethane, polyolefin-based elastomer, styrene-based elastomer, fluorine-based elastomer, or silicone-based elastomer.

In Step 5, a coating layer 40 is formed on the outer peripheral surface of the foamed material layer 36, as shown in FIG. 11. This coating layer 40, like the one according to the embodiment shown in FIGS. 6 to 8, is formed by coating the outer peripheral surface of the foamed material layer 36 with a material that has high chemical resistance and slip characteristics, e.g., urethane- or fluorine-based resin, by dipping or extrusion molding. The resin material of the coating layer 40 penetrates pores on the outer peripheral surface of the foamed material layer 36. Thus, the flexible tube 26 shown in FIG. 11 is formed.

The following is a description of functions and effects of the flexible tube 26 for endoscope according to the third embodiment constructed in this manner.

In inserting the insertion section 2 of the endoscope 1 into the body cavity, as in the case of the second embodiment, the flexible tube 26 fulfills its high resilience that is based on the high resilience of the foamed material layer 36.

Further, the continuous cell of the foamed material layer 36 is impregnated with the impregnant material that has steam barrier properties. In subjecting the endoscope 1 to autoclave sterilization, therefore, the flexible tube 26 fulfills its steam barrier properties that are based on the foamed material layer 36 impregnated with the impregnant material. Thus, the endoscope 1 can deal with the autoclave sterilization.

Further, the coating layer 40 is formed on the outer peripheral surface of the foamed material layer 36 by dipping or extrusion molding, and the resin material of the coating layer 40 penetrates the pores on the outer peripheral surface of the foamed material layer 36. With this arrangement, adhesion between the foamed material layer 36 and the coating layer 40 is improved, and the steam barrier properties are enhanced.

The following is a description of a flexible tube for endoscope according to a fourth embodiment of the invention. The same reference numerals are used to designate similar portions of the flexible tubes according to the second and fourth embodiments, and a description of those portions is omitted. Steps 1 to 3 of manufacturing processes for a flexible tube 26 according to the present embodiment are equivalent to Step 1 to 3, respectively, of the second embodiment. More specifically, a net tube 32 is fitted on a helical tube 30, a adhesive agent 35 is applied to the net tube 32, and a foamed material layer 36 is put on the adhesive agent 35. In Step 4, the outer peripheral surface of the foamed material layer 36 is melted by heating, whereupon its foamed configuration is flattened to form a substantially uniform curved surface. In Step 5, as in Step 5 of the second embodiment, the foamed material layer 36 is coated with the coating layer 40.

The following is a description of functions and effects of the flexible tube 26 for endoscope according to the fourth embodiment constructed in this manner. In inserting an insertion section 2 of an endoscope 1 into the body cavity, as in the case of the second embodiment, the flexible tube 26 fulfills its high resilience that is based on the high resilience of the foamed material layer 36. Thus, the endoscope 1 can be smoothly inserted into the body cavity.

Further, the outer peripheral surface of the foamed material layer 36 is melted by heating, whereupon its foamed configuration is flattened to form a substantially uniform curved surface. In subjecting the endoscope 1 to autoclave sterilization, therefore, the flexible tube 26 fulfills its steam barrier properties that are based on the outer peripheral surface of the foamed material layer 36. Thus, the endoscope 1 can deal with the autoclave sterilization.

The aforementioned manufacturing processes for the flexible tube 26 are a mere example, and may be modified variously only if the flexible tube 26 can be formed without a change in configuration.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

1. A flexible tube for endoscope comprising:
an outer skin layer covering an outer peripheral surface of a tubular member; and
a topcoat covering an outer peripheral surface of the outer skin layer,
wherein the outer skin layer and the topcoat are molded by substantially simultaneously laminating resin materials together.

2. A flexible tube for endoscope according to claim 1, wherein the outer skin layer and the topcoat are each kept at a temperature higher than normal temperature and lower than a fusion temperature when the outer skin layer and the topcoat are laminated.

3. A flexible tube for endoscope according to claim 2, wherein the temperature of the outer skin layer is set at a temperature close to and lower than the temperature of the topcoat when the topcoat is laminated.

4. A flexible tube for endoscope according to claim 3, wherein the tubular member has a helical tube and a net tube put on the helical tube.

5. A flexible tube for endoscope comprising:
an outer skin layer covering an outer peripheral surface of a tubular member; and
a topcoat covering an outer peripheral surface of the outer skin layer,
the outer skin layer and the topcoat being molded by successively laminating resin materials together.

6. A flexible tube for endoscope according to claim 5, wherein the outer skin layer and the topcoat are each kept at a temperature higher than normal temperature and lower than a fusion temperature when the outer skin layer and the topcoat are laminated.

7. A flexible tube for endoscope according to claim 6, wherein the temperature of the outer skin layer is set at a temperature close to and lower than the temperature of the topcoat when the topcoat is laminated.

8. A flexible tube for endoscope according to claim 7, wherein the tubular member has a helical tube and a net tube put on the helical tube.

9. A method for manufacturing a flexible tube for endoscope, comprising:
laminating an outer skin layer of a resin material to an outer peripheral surface of a tubular member; and
laminating a topcoat of a resin material to an outer peripheral surface of the outer skin layer,
wherein laminating of the outer skin layer and the topcoat are performed substantially simultaneously.

10. The method according to claim 9, wherein the outer skin layer and the topcoat are each kept at a temperature higher than normal temperature and lower than a fusion temperature when the outer skin layer and the topcoat are laminated.

11. The method according to claim 10, wherein the temperature of the outer skin layer is set at a temperature close to and lower than the temperature of the topcoat when the topcoat is laminated.

12. A method for manufacturing a flexible tube for endoscope, comprising:
laminating an outer skin layer of a resin material to an outer peripheral surface of a tubular member; and
laminating a topcoat of a resin material to an outer peripheral surface of the outer skin layer,
wherein laminating of the outer skin layer and the topcoat are performed successively.

13. A method according to claim 12, wherein the outer skin layer and the topcoat are each kept at a temperature higher than normal temperature and lower than a fusion temperature when the outer skin layer and the topcoat are laminated.

14. A method according to claim 13, wherein the temperature of the outer skin layer is set at a temperature close to and lower than the temperature of the topcoat when the topcoat is laminated.

15. A flexible tube for endoscope comprising:
a helical tube;
a net tube put on the helical tube;
an outer skin put on an outer peripheral surface of the net tube; and
a topcoat put on a surface of the outer skin,
wherein the outer skin and the topcoat are molded substantially simultaneously or successively by the two-coat molding method.

16. The flexible tube for endoscope according to claim 15, wherein the outer skin contains a thermoplastic elastomer.

17. The flexible tube for endoscope according to claim 15, wherein the topcoat contains a fully-saturated styrene-based thermoplastic elastomer or an elastomer obtained by copolymerizing the fully-saturated styrene-based thermoplastic elastomer and isobutylene.

18. The flexible tube for endoscope according to claim 15, wherein the net tube and the outer skin are bonded together with a urethane-based adhesive agent.

19. A method for manufacturing a flexible tube for endoscope, comprising:
spirally winding a thin elastic sheet to form a helical tube;
putting a net tube on the helical tube; and
welding and molding two types of resins substantially simultaneously on an outer peripheral surface of the net tube, thereby forming an outer skin and a topcoat welded together.

20. The method according to claim 19, wherein the outer skin contains a thermoplastic elastomer.

21. The method according to claim 19, wherein the topcoat contains a fully-saturated styrene-based thermoplastic elastomer or an elastomer obtained by copolymerizing the fully-saturated styrene-based thermoplastic elastomer and isobutylene.

22. The method according to claim 19, wherein the net tube and the outer skin are bonded together with an urethane-based adhesive agent.
